SEQUENCE LISTING (5 sequences)

SEQ ID NO: 1 - DNA, 76 bp, synthetic construct
  misc_feature 1..76: note = Apt-2019 Aptamer
  source 1..76: mol_type = other DNA
atccagagtgacgcagcagaatgggtgggattcctcagaggggttggtgagtttgtgatggacacggtggcttagt

SEQ ID NO: 2 - DNA, 76 bp, synthetic construct
  misc_feature 1..76: note = Apt-1194 Aptamer
  source 1..76: mol_type = other DNA
atccagagtgacgcagcaccgagcgagttggtttgctgcggtgggcagagaggtggggtggacacggtggcttagt

SEQ ID NO: 3 - DNA, 76 bp, synthetic construct
  misc_feature 1..76: note = Apt-1798 Aptamer
  source 1..76: mol_type = other DNA
atccagagtgacgcagcaatcgtggacctggctgtggtttgctgaggtgggcgccaattggacacggtggcttagt

SEQ ID NO: 4 - DNA, 50 bp, synthetic construct
  misc_feature 1..50: note = Apt-NG2019 (14-63) Aptamer
  source 1..50: mol_type = other DNA
cagcagaatccgtgccattcctcagaccggttcctgagtttgtgatggac

SEQ ID NO: 5 - DNA, 58 bp, synthetic construct
  misc_feature 1..58: note = apt-ng1194 (1-58) aptamer
  source 1..58: mol_type = other DNA
atccagagtgacgcagcaccgagcgagttggtttgctgccctgcccagagacctggcc

(19) United States
(12) Patent Application Publication
(10) Pub. No.: US 2025/0108137 A1
(43) Pub. Date: Apr. 3, 2025

(54) APTAMERS SPECIFIC TO EBV-ASSOCIATED MALIGNANCIES AND THEIR APPLICATIONS IN CANCER IMAGING AND THERAPIES

(71) Applicant: The Chinese University of Hong Kong, Hong Kong (CN)

(72) Inventors: Anna Chi Man TSANG, Hong Kong (CN); Yongshu LI, Hong Kong (CN); Kwok Wai LO, Hong Kong (CN)

(21) Appl. No.: 18/833,184

(22) PCT Filed: Feb. 8, 2023
(86) PCT No.: PCT/CN2023/075031

§ 371 Date: Jul. 25, 2024

Related U.S. Application Data
(60) Provisional application No. 63/267,737, filed on Feb. 9, 2022.

(51) Int. Cl. A61K 51/04; A61K 47/54; A61P 35/00; C12N 15/115

(52) U.S. Cl. CPC A61K 51/0491; A61K 47/549; A61P 35/00; C12N 15/115; A61K 2123/00; C12N 2310/16

(57) ABSTRACT

Provided are aptamers and compositions thereof for use in methods for imaging and treating cancer. The aptamers can bind to targets including CCHC-Type Zinc Finger Nucleic Acid Binding Protein (CNBP) and metadherin (MTDH). The aptamers can be conjugated to various pharmaceutical drugs and/or biocompatible imaging probes.

### CROSS-REFERENCE TO RELATED APPLICATION

This application claims the benefit of U.S. Patent Application Ser. No. 63/267,737, filed Feb. 9, 2022, which is hereby incorporated by reference in its entirety including any tables, figures, or drawings.

### BACKGROUND OF THE INVENTION

In recent years, proof-of-concept studies in aptamer technology continue to reveal their promising functionality and vast therapeutic potential (1, 2). Aptamers can enhance both the targeting specificity and pharmacokinetic profile of the conjugated drugs or nanocarriers. To date, one aptamer has been approved by the U.S. Food and Drug Administration (FDA) and ten aptamers have entered into clinical trials for the treatment of solid tumors, lymphoma, macular degeneration, coagulation, and inflammation (2).

Aptamers are short single-stranded oligonucleotides (e.g. DNA, RNA) that bind to desirable targets with high affinity and specificity by folding into tertiary structures. To obtain aptamers which can specifically bind to a target, Systematic Evolution of Ligands by EXponential enrichment (SELEX) (3, 4) of a small subset of aptamers from a ssDNA library to a given target is typically performed. This allows for the simultaneous screening of 1×10¹⁵ different 40-nucleotide aptamers against a target of interest by 10-15 rounds of positive PCR enrichment. Almost all aptamers selected through SELEX exhibit high affinity to targets with a dissociation constant (K_{d}) in micromolar to nanomolar range. Therefore, aptamers are also called “chemical antibodies” and have quickly emerged as a novel and powerful class of ligands with excellent potential for molecular probes development, biosensor, tumor biomarker discovery and drug-delivery (3, 5-7). Cell-SELEX is a modified SELEX process, where live cells are used as target for aptamers selection (4). Compared with traditional selections that target purified proteins, Cell-SELEX has enabled the generation of aptamers that specifically bind a target molecule on cell surface in its native conformation. In vivo-SELEX can further enrich the aptamers that can be accumulated in the tumor in mice model.

Nasopharyngeal carcinoma (NPC) is a squamous cell epithelial malignancy arising in the nasopharynx (8, 9). The peak age at diagnosis is in the upper 40s to 50s. In Hong Kong, NPC is the top cancer in term of incidence among the males below the age of 40 (4). Therefore, this endemic disease places socio-economic burdens to these relatively young cancer patients and their families.

Concurrent chemoradiation therapy remains as the mainstay treatment of primary NPC. Moreover, due to the hidden location of the nasopharynx and non-specificity of initial symptoms, many NPC cases are only diagnosed late at stages III or IV (10). To aid the diagnosis, imaging probes which can visualize the primary and metastatic cancer sites is essential.

Moreover, whenever systemic chemotherapy being applied in primary, recurrent and metastatic cases, patients may suffer from acute toxicity including hearing loss, swallowing problems and others; and late toxicity including cranial neuropathy, temporal lobe necrosis, aspiration pneumonia etc. More effective and less-toxic treatment regimens are always in need for better caring of NPC patients (8, 10).

In the last decade, we and others have strived for translational genomic to search for druggable targets in NPC (11-15). However, druggable molecular targets such as PIK3CA, EGFR, FGFR1/2/3/4 and BRCA1/BRCA2/ATM are not common in NPC, and only occur in a total of 6% patients. This low mutational load in NPC restrains the potential of targeted therapies relying on approved drugs available immediately.

The undifferentiated nonkeratinizing subtype of NPC, commonly seen in South China and Hong Kong, is almost universally associated with EBV infection (8, 16). In NPC cells, EBV hides in a latent state and expresses only two latent membrane proteins (LMP1 and LMP2), intracellular RNAs (EBERs and EBV-microRNAs), and a nuclear protein (EBNA1) for EBV maintenance and NPC development. LMP1 remains as the major oncogenic protein in the carcinogenesis of NPC (17, 18). Around 30% of NPC tumors express high levels of LMP1 and are correlated with poor disease outcome and less genomic mutations (12). We have also proposed a model of the clonal evolution of NPC driven by EBV infection and progressive genomic changes in precancerous nasopharyngeal epithelium (11, 16, 18).

Thus, there remains a need for aptamers that can specifically target to the cell surface markers in Epstein-Barr virus (EBV)-associated NPC.

### BRIEF SUMMARY OF THE INVENTION

The present invention relates to aptamers and compositions thereof for use in methods for imaging cancer cells and treating cancer. The present disclosure provides the nucleic sequences of aptamers according to SEQ ID NO: 1, 2, or 3 or an oligonucleotide having at least 90% identity to SEQ ID NO: 1, 2, or 3. In certain embodiments, the aptamers can bind to targets including, for example, CCHC-Type Zinc Finger Nucleic Acid Binding Protein (CNBP) and metadherin (MTDH). In certain embodiments, the aptamers can enable cancer imaging by linking the aptamers with molecular probes, such as, for example, fluorescent probes and positron emission tomography radiotracers. In certain embodiments, the aptamers can be used for drug targeting by loading the aptamers with said drug, such as, for example, toxic nucleotide analogues as well as other anti-cancer nanoparticles.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1B. Discovery of Apt-2019 targeting to LMP1-expressing cells. (FIG. 1A) Enriching of aptamers with higher binding affinity to LMP1-expressing cells after 12 rounds of Cell-SELEX. The enrich pools of aptamers were sequenced and clustered by similarity. 8 aptamers were selected for FACS analysis of binding affinity to LMP1-expressing cells. (FIG. 1B) Specificity of Apt-2019 in binding with LMP1-positive cells was validated by FACS analysis (left panel) and fluorescence imaging (right panel).

FIGS. 2A-2B. CNBP was identified to be the binding target of Apt-2019. (FIG. 2A) Schematic diagram showing the procedures in identifying the binding partner of Apt-2019. (FIG. 2B) CNBP was purified and linked to beads. After binding with FAM-tagged Apt-2019, high fluorescence signal was only emitted from beads tagged with CNBP.

FIGS. 3A-3H. Apt-2019 was identified as a CNBP-binding aptamer on LMP1-expressing cells. (FIG. 3A) Using non-permeabilized cells, fluorescent microscopy showed the cell surface stainings of Apt-2019 and CNBP on LMP1-expressing NPC43 (noEBV) cells. Notably, the expression of LMP1 in NPC cells could induce the expression of CNBP, which could be bound with the Apt-2019. (FIG. 3B) LMP1 expression could upregulate the expression of CNBP. (FIG. 3C) The binding of Apt-2019 was much lower in the CNBP-knockout cells compared to the parental cells. (FIG. 3D) The membranous, cytoplasmic and nuclear protein lysates were extracted from the parental and CNBP-knockout cells. CNBP was abundantly expressed in the membrane fraction. (FIG. 3E) Overexpression of CNBP was found in NPC cell lines and patent-derived xenografts. Primary normal nasopharyngeal epithelial cells were immortalized by hTert (catalytic domain of telomerase). (FIG. 3F) High expression of CNBP was detected in primary NPC tissue and xenograft-76 by IHC. The higher magnification image shows the abundant membranous expression of CNBP. (FIG. 3G) Knockdown of CNBP by siRNAs could suppress the proliferation in NPC cells. (FIG. 3H) Would healing assay was used to assess the migration ability of NPC cells. Knockdown of CNBP could suppress the migration of NPC cells.

FIGS. 4A-4F. Apt-1194 & Apt-1798 were identified as a targeting aptamers to EBV-positive cell lines. (FIG. 4A) Schematic diagram showing the procedures in discovery of aptamers targeting to EBV-positive NPC tumor by a combination of Cell-SELEX and In vivo-SELEX. The aptamers which bind preferentially to EBV-positive cells were enriched by 12 rounds of Cell-SELEX. This aptamer pool was then injected to mice and continued for two rounds of In vivo-SELEX. The enriched aptamers were then subjected to sequencing. Apt-1194 & Apt-1798 were the top enriched sequences. (FIG. 4B) Apt-1194 & Apt-1798 could bind specifically to EBV-positive cell lines but not to EBV-negative cell lines (A549 is a lung cancer cell line; NP460 is an immortalized NP cell line). (FIG. 4C) The Apt-1194 or Apt-library were used to pull down proteins from EBV-positive NPC43 cells. The differentially pulled protein was purified and identified as MTDH by mass spectrometry and western blot analysis. (FIG. 4D) High expression of MTDH was detected in primary NPC tissue and xenograft-76 by IHC. The higher magnification image shows the abundant membranous expression of MTDH. (FIG. 4E) Overexpression of MTDH were found in NPC cell lines and patent-derived xenografts. (FIG. 4F) Knockdown of MTDH could suppress the proliferation and migration in NPC cells.

FIGS. 5A-5G. Characterization of Apt-2019 and Apt-1194. (FIG. 5A) Diagram showing the G-quadraplex structure that can be formed by G-rich aptamers. (FIGS. 5B-5C) Fluorescent enhancement assay (FEA) was used to detect G-quadraplex forming sequences. Both Apt-2019 and Apt-1194 could form into G-quadraplex in the presence of K+ or L+ ions and then light up the ThT ligands. (FIG. 5D) Apt-2019 and Apt-1194 displayed a much higher serum stability than random aptamer sequences. (FIGS. 5E-5F) A serial concentrations of FITC-tagged Apt-2019 and Apt-1194 were bound with LMP1-overexpressing NPC43 and EBV-positive C666-1 respectively. Equilibrium dissociation constant (Kd) was then calculated. (FIG. 5G) Apt-2019 was allowed to bind with live NPC cells for 45 mins. Internalization of Apt-2019 was validated by Z-stack imaging of confocal microscope.

FIGS. 6A-6B. Apt-2019 and Apt-1194 could be specifically targeted to Xenograft-C15 and C666-1 tumors respectively. Aptamers were labelled with fluorescence to enable imaging of their biodistribution in mice models. In the control aptamers (Apt-NG), all the G nucleotides were replaced with C nucleotides to destroy the G-quadraplex 3-D conformation. (FIG. 6A) Accumulation of Apt-2019, but not its control counterpart, could be observed in the C15 xenograft. (FIG. 6B) Apt-1194 could also be selectively targeted to the EBV+ve tumor if comparing to the Apt-NG1194 control aptamer. Detection of fluorescent signals in liver is not unexpected, as liver is the site for detoxifying and breaking down unwanted substances.

FIGS. 7A-7E. Cytotoxicity of 5FU-loaded Apt-2019 and gemcitabine-loaded Apt-1194. (FIG. 7A) Intrinsic incorporation of aptamers with gemcitabine or 5-FU. (FIGS. 7B-7C) The binding affinity of drug-free and drug-loaded Apt-2019 and Apt-1194 on target cells. Both the 5-FU-and gemcitabine-incorporated aptamers could maintain the binding affinity. (FIGS. 7D-7E). In vitro toxicity of drug-loaded aptamers to target cells. Apt-2019-5FU and Apt-1194-gem could suppress the growth of LMP1+ve and EBV+ve NPC cells respectively.

FIGS. 8A-8D. Loading the 5-FU into the Apt-2019 could greatly downregulate the effective treatment dose by >10 folds. (FIG. 8A) Mice bearing Xeno113 were treated with DPBS, Free 5-FU and Apt-2019-5FU via tail vein injection. Apt-2019 contains 13 Thymidine nucleotides, and we have exchanged them all to 5-FU. To demonstrate the Apt-2019 could enhance the delivery of 5-FU into the targeted tumor, we have used a dose of 5-FU which is 11.5 folds less than the normal treatment dose. Same molar concentration of 5-FU was used in the free 5-FU and Apt-2019-5FU treatment groups. The free 5-FU could not significantly suppress the tumor growth, while the Apt-2019-5FU could suppress the tumor growth. There is no observable change in the (FIG. 8B) morphology of spleen, liver and kidney and (FIG. 8C) body weight of mice between the control and treatment groups. (FIG. 8D) Apt-2019-5FU showed potent inhibitory effect of the growth of XenoC15, which is known to have high expression of LMP1.

### BRIEF DESCRIPTION OF THE SEQUENCES

SEQ ID NO: 1: Apt-2019 Aptamer

SEQ ID NO: 2: Apt-1194 Aptamer

SEQ ID NO: 3: Apt-1798 Aptamer

SEQ ID NO: 4: Apt-NG^{2019 (14-63)} Aptamer

SEQ ID NO: 5: Apt-NG^{1194 (1-58)} Aptamer

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to administer aptamers or to treat and/or image cancerous cells. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), thiol, digoxigenin, ³²P and other isotopes, haptens, and proteins which can be made detectable, e.g., by conjugating a radiolabel to the aptamer. The term includes combinations of single labeling agents, e.g., a combination of fluorophores that provides a unique detectable signature, e.g., at a particular wavelength or combination of wavelengths. In the context of detecting nucleic acids (e.g., target sequences), the aptamers can, typically, be labeled with radioisotopes, fluorescent labels (fluorophores), or luminescent agents.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a nucleotide aptamer used in the method of this invention has at least 70% sequence identity, preferably 80%, 85%, 90%, 91%, 92%, 93%, 94%, 95%, 96%, 97%, 98%, 99%, or 100% identity, to a different aptamer sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Preparation of Aptamers and Compositions Thereof

In certain embodiments, one or more aptamers that bind to a target nucleic acid or protein, including, for example, CCHC-Type Zinc Finger Nucleic Acid Binding Protein (CNBP) and/or metadherin (MTDH), is provided by the subject invention.

Typically, the aptamers can be at least 10 bases, more often at least about 15, about 20,about 25, about 30, about 35, about 40, about 45, about 50, about 55, about 60, about 65,about 70, about 75, about 80, about 85, about 90, about 95, about 100, about 110, about 120,about 130, about 140, about 150, or more base pairs in length.

In preferred embodiments, one, two, three, four, five, six, seven, eight, nine, ten, or more aptamers can be used in the subject compositions and methods. In certain embodiments, the aptamers can be according to SEQ ID NO: 1 (Apt-2019 Aptamer: ATCCAGAGTGACGCAGCAGAATGGGTGGGATTCCTCAGAGGGGTTGGTGAGTTT GTGATGGACACGGTGGCTTAGT), SEQ ID NO: 2 (Apt-1194 Aptamer: ATCCAGAGTGACGCAGCACCGAGCGAGTTGGTTTGCTGCGGTGGGCAGAGAGGT GGGGTGGACACGGTGGCTTAGT), SEQ ID NO: 3 (Apt-1798 Aptamer: ATCCAGAGTGACGCAGCAATCGTGGACCTGGCTGTGGTTTGCTGAGGTGGGCGC CAATTGGACACGGTGGCTTAGT) or comprise a sequence having at least about 75%, about 80%, about 85%, about 90%, about 95%, about 96%, about 97%, about 98%, about 99%, or about 100% identity to SEQ ID NO: 1, SEQ ID NO: 2, SEQ ID NO: 3.

In certain embodiments, the aptamers herein can include any useful label, including a thiol group, a biotin, or an enzyme, or fluorescent labels and quencher labels at any useful position in the nucleic acid sequence, such as, for example at the 3′- and/or 5′-terminus. Exemplary fluorescent labels include a quantum dot or a fluorophore. Examples of fluorescence labels for use in this method includes fluorescein, 6-FAM™ (Applied Biosystems, Carlsbad, Calif.), TET™ (Applied Biosystems, Carlsbad, Calif.), VIC™ (Applied Biosystems, Carlsbad, Calif), MAX, HEX™ (Applied Biosystems, Carlsbad, Calif), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rhol01, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, ABY™ (Applied Biosystems, Carlsbad, Calif.), TAMRA™ (Applied Biosystems, Carlsbad, Calif.), 5-TAMRA, JUN™ (Applied Biosystems, Carlsbad, Calif.), ROX™ (Applied Biosystems, Carlsbad, Calif.), Oregon Green® (Life Technologies, Grand Island, N.Y.), Oregon Green 500, IRDye® 700 (Li-Cor Biosciences, Lincoln, Nebr.), IRDye 800, WeIIRED D2, WeIIRED D3, WeIIRED D4, and Lightcycler® 640 (Roche Diagnostics GmbH, Mannheim, Germany). In some embodiments, bright fluorophores with extinction coefficients >50,000 M⁻¹ cm⁻¹ and appropriate spectral matching with the fluorescence detection channels can be used.

In certain embodiments, a fluorescently labeled aptamer is included in a reaction mixture and a fluorescently labeled reaction product is produced. Fluorophores used as labels to generate a fluorescently labeled aptamer included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumarin 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl} amino)naphthalene-1-sulfonic acid), Malachite Green isothiocyanate; 4-methylumbelliferone; orthocresolphthalein; nitrotyrosine; pararosaniline; Phenol Red; B-phycocrytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid; and terbium chelate derivatives.

Exemplary quencher labels include a fluorophore, a quantum dot, a metal nanoparticle, and other related labels. Suitable quenchers include Black Hole Quencher®-1 (Biosearch Technologies, Novato, CA), BHQ-2, Dabcyl, Iowa Black® FQ (Integrated DNA Technologies, Coralville, IA), IowaBlack RQ, QXL™ (AnaSpec, Fremont, CA), QSY 7, QSY 9, QSY 21, QSY 35, IRDye QC, BBQ-650, Atto 540Q, Atto 575Q, Atto 575Q, MGB 3′ CDP13, and MGB-5′ CDP13. In one instance, the term “quencher” refers to a substance which reduces emission from a fluorescent donor when in proximity to the donor. In preferred embodiments, the quencher is within 10, 11, 12, 13, 14, 15, 16, 17, 18, 19, 20, 21, 22, 23, 24, 25, 26, 27, 28, 29 or 30 nucleotide bases of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, the concentration of the aptamer in the compositions and method of use is about 1 pM to about 1 M, about 1 nM to about 10 mM, about 0.01 μM to about 100 μM, about 0.1 μM to about 100 μM, about 0.1 μM to about 50 μM, about 0.1 μM to about 20 μM, or about 1 μM to about 20 μM.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or car.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays arc, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propanc, nitrogen, fluorocarbons, and/or other solubilizing or dispersing agents known in the art. Illustratively, delivery may be by use of a single-use delivery device, a mist nebulizer, a breath-activated powder inhaler, an acrosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono-or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Administration of Aptamers

In certain embodiments, an aptamer can be administered to a subject. Any means of administration that can permit an aptamer to contact cells in a subject, particularly cancerous or tumor cells, including, for example, orally, intravenously intranasally, intraperitoneally, intramuscularly, intrathecally, or subcutaneously are envisioned in the subject methods. In preferred embodiments, an aptamer can be administered intravenously.

In certain embodiments, an aptamer can contact healthy cells of subject and/or tumor cells or cancerous cells. In certain embodiments, an aptamer can contact cells in the nasopharynx, including cancerous cells of the nasopharynx. In certain embodiments, an aptamer can inhibit nasopharyngeal carcinoma, pancreatic cancer, gastric carcinoma, and breast cancer, and other solid tumors. The aptamer can bind to proteins on the surface of the cells of a subject, including, for example, cancerous cells. In certain embodiments, the proteins can be CNBP and/or MTDH. In certain embodiments, the aptamers can be conjugated to pharmaceuticals, including, for example, nucleoside analogues, or biocompatible imaging probes. In certain embodiments, the aptamer-pharmaceutical conjugate can inhibit the growth of tumor cells.

### Use of Aptamers as Molecular Probes for Visualizing In Vivo Tumors

Early detection, accurate diagnosis and prediction of disease progression are crucial for optimal treatment planning. In current clinical practice, Positron emission tomography (PET)-computed tomography (PET-CT) using 18F-FDG provides combined anatomic and metabolic information in tumor examination.

In certain embodiments, the aptamers, specifically the aptamers according to SEQ ID NO: 1, 2, or 3 or having at least 90% identity to SEQ ID NO: 1, 2, or 3, can be tagged with a variety of biocompatible imaging probes, such as, for example, radioactive tracers compatible with PET scanning. The tagging of aptamers has been previously described by Jacobson O et al., PET imaging of tenascin-C with a radiolabeled single-stranded DNA aptamer. J Nucl Med. 2015 April; 56 (4):616-21. doi: 10.2967/jnumed.114.149484. Epub 2015 Feb. 19. PMID: 25698784, which is hereby incorporated by reference in its entirety. Jacobsen et al. describe the radiosynthesis of the 18F-Fluorobenzoyl (FB) aptamer and the 64Cu-NOTA aptamer and the synthesis of 18F-fluoride labelled N-succinimidyl 4-fluorobenzoate (18F-SFB) and 64Cu labelled S-2-(4-isothiocyanate benzyl)-1,4,7-triazacyclononane-1,4,7-triacetic acid (64Cu-SCN-Bn-NOTA) before conjugating them with the aptamers. In certain embodiments radio tracers than can be used include, for example, tritium, carbon-11, carbon-14, oxygen-15, fluorine-18, copper-64, phosphorus-32, sulfur-35, technetium-99, iodine-123, and gallium-67, antimony-124, bromine-82, iodine-125, iodine-131, iridium-192, and scandium-46, manganese-56, sodium-24, technetium-99m, silver-110m, argon-41, and xenon-133. In certain embodiments, the biocompatible imaging probe can be 18F-FDG, 64Cu-NOTA, 11C-methionine, or 11C-choline. In certain embodiments, the aptamers tagged with one or more imaging probes can be used in methods of identifying tumor cells, such as, for example, EBV/LMP1-positive tumors cells as well as other cancer cell types that show an overexpression of the aptamer-targeting surface molecules. In certain embodiments, PET can be used as a nuclear imaging test for identifying the tumor cells. The PET scan can integrate with computed tomography (CT) and radioactive tracer-labelled aptamers. Before the PET scan, the aptamers (labelled with radioactive tracers) can be injected into the bloodstream of a subject. After the tracer is absorbed in the subject, the subject can be positioned in the PET scanner. The tracer is radiolabeled, meaning it emits gamma rays that are detected by the PET scanner. The computer collects the information emitted by the tracer and displays it on the CT cross-sections for identifying the tumor cells which have higher accumulation of the tracer-labelled aptamers. In certain embodiments, the aptamer-targeting surface molecules are CNBP and/or MTDH.

In certain embodiments, the other cancer cell types that show overexpression of the aptamer-targeting surface molecules, include, for example, nasopharyngeal carcinoma, pancreatic cancer, gastric carcinoma, bladder cancer, colon and rectal cancer, endometrial cancer, kidney cancer, leukemia, liver cancer, lung cancer, melanoma, and breast cancer.

### Aptamer-Based Delivery of Chemodrugs and Nanoparticles as Effective Therapeutic Strategy for Cancers

In certain embodiments, a pharmaceutical compound or drug can be conjugated to the aptamers, specifically aptamers according to SEQ ID NO: 1, 2, or 3 or having at least 90% identity to SEQ ID NO: 1, 2, or 3. In certain embodiments, compound can be either tagged at the 5′ or 3′ ends of the aptamers. In certain embodiments, when toxic nucleoside analogues are used, such as, for example, gemcitabine or Fluorouracil, these analogues can be exchanged with nucleotides of the aptamers. In certain embodiments, the drug can be a toxic nucleoside analogue, such as, for example, gemcitabine, Fluorouracil (5-FU), cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine (dideoxyinosine: ddI), zalcitabine (dideoxycytinc: ddC), stavudine (d4T), or zidovudine (AZT). In certain embodiments, aptamers can be conjugated to a nanoparticle, such as, for example, dendrimers or lipid-based nanoparticles. In certain embodiments, the nanoparticle or dendrimer can contain anti-tumor chemicals, such as, for example, siBCL3, siPRMT5, and siMAT2A; siRNAs, such as, for example, siBCL3, siPRMT5, and siMAT2A; antibodies, such as, for example, Alemtuzumab, Atezolizumab, Avelumab, Bevacizumab, Blinatumomab, Brentuximab, Cemiplimab, Cetuximab, Daratumumab, Dinutuximab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Tositumomab, and Trastuzumab; alkylating agents, such as, for example, Altretamine, Bendamustine, Busulfan, Carmustine, Chlorambucil, Cyclophosphamide, Dacarbazine, Ifosfamide, Lomustine, Mechlorethamine, Melphalan, Procarbazine, Streptozocin, Temozolomide, Thiotepa, and Trabectedin; platinum coordination complexes, such as, for example, Carboplatin, Cisplatin, and Oxaliplatin; antibiotics, such as, for example, Bleomycin, Dactinomycin, Daunorubicin, Doxorubicin, Epirubicin, Idarubicin, Mitomycin, Mitoxantrone, Plicamycin, and Valrubicin; antifolates, such as, for example, Methotrexate, Pemetrexed, Pralatrexate, and Trimetrexate; purine analogues, such as, for example, Azathioprine, Cladribine, Fludarabine, and Mercaptopurine; pyrimidine Analogues, such as, for example, Azacitidine, Capecitabine, Cytarabine, Decitabine, Floxuridine, Fluorouracil, Gemcitabine, and Trifluridine/Tipracil; biologic response modifiers, such as, for example, Aldesleukin (IL-2), Denileukin Diftitox, and Interferon Gamma; histone deacetylase inhibitors, such as, for example, Belinostat, Panobinostat, Romidepsin, and Vorinostat; Antiandrogens, such as, for example, Abiraterone, Apalutamide, Bicalutamide, Cyproterone, Enzalutamide, Flutamide, and Nilutamide; Antiestrogens (including Aromatase Inhibitors), such as, for example, Anastrozole, Exemestane, Fulvestrant, Letrozole, Raloxifene, Tamoxifen, and Toremifene; Gonadotropin Releasing Hormone Analogues, such as, for example, Degarelix, Goserelin, Histrelin, Leuprolide, and Triptorelin; Peptide Hormones, such as, for example, Lanreotide, Octreotide, and Pasircotide; Protein Kinase Inhibitors, such as, for example, Abemaciclib, Acalabrutinib, Afatinib, Alectinib, Alpelisib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Gefitinib, Gilteritinib, Glasdegib, Ibrutinib, Idelalisib, Imatinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pexidartinib, Ponatinib, Regorafenib, Ribocicib, Rucaparib, Ruxolitinib, Selumetinib, Sonidegib, Sorafenib, Sunitinib, Talazoparib, Trametinib, Vandetanib, Vemurafenib, Vismodegib, and Zanubrutinib; Topoisomerase Inhibitors, such as, for example, Etoposide, Irinotecan, Teniposide, and Topotecan; Vinca Alkaloids, such as, for example, Vinblastine, Vincristine, and Vinorelbine; and/or other compounds, including, for example, Asparaginase (Pegaspargase), Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, and/or Venetoclax. In certain embodiments, some drugs contain a thiol group, such as, for example, 6-Mercaptopurine (anticancer), Captopril (antihypertensive), D-penicillamine (antiarthritic), Sodium aurothiolate (antiarthritic) and can be used to conjugate with the aptamers. In certain embodiments, the aptamer conjugate can be used to deliver pharmaceuticals, including those within a nanoparticle, to specific location within a subject, such as, for example, to a cancerous cell.

### MATERIALS AND METHODS

### Nonmalignant and Cancerous Nasopharyngeal Epithelial Cell Lines

NP460 cells were cultured in the 50% complete Eplife medium (Thermo Fisher Scientific) and 50% complete Defined Keratinocyte-SFM (Thermo Fisher Scientific) with 100 unit/ml penicillin and 100 μg/ml streptomycin. NPC43 LMP1+ve and LMP1−ve cells were cultured in RPMI-1640 (Sigma) with 10% fetal bovine serum, 4 μM Y27632 dihydrochloride (Alexis), 100 unit/ml penicillin, and 100 μg/ml streptomycin. HONE1-LMP1+ve cells were cultured in RPMI-1640 (Sigma) with 10% fetal bovine serum, 100 unit/ml penicillin, and 100 μg/ml streptomycin. NPC43-EBV+ve and NPC43-EBV−ve cells were cultured in RPMI-1640 (Sigma) with 10% fetal bovine serum, 4 μM Y27632 dihydrochloride (Alexis), 100 unit/ml penicillin, and 100 μg/ml streptomycin. C666-1 and A549 cells were cultured in RPMI-1640 (Sigma) with 10% fetal bovine serum, 100 unit/ml penicillin, and 100 μg/ml streptomycin.

### NPC Xenografts

Four to five weeks old male immune deficient mice (NOD/SCID) were supplied by the Laboratory Animal Unit (LAU) of The Chinese University of Hong Kong and housed under pathogen-free conditions. All animal experiments were conducted in conditions according to the animal license issued from the Hong Kong Department of Health and with the approval of the Animal Experimentation Ethics Committee (AEEC) of the Chinese University of Hong Kong. To initiate growth of NPC43-LMP1+ve and LMP1−ve cell lines as tumor xenografts in mice, 1×10⁷ cells were resuspended in 200 μl (1:1 mixture of Matrigel and culture medium) and injected subcutaneously at the left side of the dorsal flank region of each NOD/SCID mouse. For the C15 tumor xenografts, the xenografted tumors were cut into semi-solid condition and mixed with Matrigel in 1:1 ratio, then implanted into the right side of the dorsal flank region of NOD/SCID mice. Mice were then randomized into drug treatment groups or control groups once the tumors become palpable (with a diameter of 4-6 mm). The Apt-2019-5FU (2 μmol/kg) was dissolved in 200 μl DPBS containing 5 mM MgCl2 and given to animals by tail vein injection every two days. 5-FU was dissolved to 100 mM as stock concentration and diluted with DPBS for tail vein injection every two days. Tumor size and animal body weight were recorded every day during the entire treatment period. All the mice were euthanized at the end of experiment and their tumors were excised and fixed with 10% neutral buffered formalin (NBF) for histopathological examination and immunohistochemistry study.

### Cell Viability Determination

NPC cells were incubated in 100 μl culture medium at 1000 cells per well in a 96-well plate overnight. Apt-2019-5FU and 5-FU were dissolved in cell culture medium in doses ranging from 0-1 μM and added into NPC cells for 72 hrs. Cell Counting Kit 8 was use for detecting cell viability by following the manufacturer's instructions.

### Western Blot

ProteinExt® Mammalian Total Protein Extraction Kit from Transgen (DE101-01) was used to lyse the cells for Western blotting analysis. ProteinSafe™ Protease Inhibitor Cocktail, EDTA-free (100×) (Transgen, DI101-01) was included in the protein extraction kit immediately before use. Lysed protein samples were adjusted to equal protein concentration and separated by SDS-polyacrylamide gel electrophoresis. NC membranes (GE Healthcare, 0.45 μm) were used to transfer the resolved proteins in the gel. The chemiluminescence signal was captured using the ChemiDoc MP Imaging System (Bio-Rad) and detected by various antibodies against various proteins including GAPDH (1:10000; Transgen, HC301-01), CNBP (1:3000; Abclonal, 15110), MTDH (1:1000 Abclonal, A5887) and LMP1 (1:3000;Dako, M0897).

### Cell-SELEX

Cell-SELEX was performed by following the protocol of ‘Development of DNA aptamers using Cell-SELEX’ with a few modifications. In brief, the UNIQ-10 Spin Column Oligo DNA Purification Kit (sangon B511143) was used for extracting and purifying ssDNA after PCR amplifications. Aptamer pools from round 9 to round 13 were used as templates for preparing sequencing library by PCR. The libraries were sent to GuangZhou IGE company for next generation sequencing. The sequences appearance times were counted in each library and then listed based on the appearance times from high to low. Top 100 sequences from each round were analyzed in ClustalW (see worldwide website: genome.jp/tools-bin/clustalw). 8 sequences were picked out for further assay.

### Flow Cytometry

10 μM aptamers or aptamer pools were dissolved in DBPS with 5 mM MgCl2 as stock and diluted with binding buffer (DBPS with 5 mM MgCl2, 0.1% BSA, 100 mg tRNA and 0.45% glucose) into 200 nM, followed by heating at 95° C. to 5 mins and chilling on ice for 5 mins. Cells were trypsinized and incubated in the aptamer solution at 4° C. for 30-60 mins. After incubation, the cells were washed once by precooled binding buffer. At last, the samples were analyzed on BD flowcytometry. For flow cytometric analysis of bead assay, the NTA beads were incubated with CNBP-his or MTDH-his containing cell lysates or control lysates for 1 hr at 4° C. and then washed with binding buffer for 3 times. After that, the beads were incubated with FAM-Apt-2019 or FAM-Apt-1194 (200 nM) for 30 mins at 4° C. At last, the bead-aptamer complexes were washed 3 times with binding buffer and analyzed by BD flow cytometer.

### Confocal Microscopic Imaging

In live cell imaging, the NPC43-LMP1+ and NPC43-LMP1− cells were seeded in 3.5 cm confocal dishes 24 hrs before. The cells were washed with PBS 2 times and then incubated with 200 nM aptamers in binding buffer for 30 mins and washed with DPBS twice after. The Zeiss LSM880 was used for taking confocal images. In immunofluorescence and aptamer co-staining, the cells were fixed by 4% PFA for 15 mins and then blocked with 3% BSA for 1 hr. CNBP antibody was incubated with the cells in 1:100 dilution (Abclonal, 15110) for 1 hr followed with anti-Rabbit Alex-555 antibody (1:500) for 1 h. After washing for 3 times, FAM-Apt-2019 was added and incubated for 1 h.

### Pulldown Assay and Mass Spectrometry

The pulldown assay was performed by following the protocol from Shang Guan's lab [235-237]. Then the protein samples were separated in PAGE gels and stained by pierce silver staining kit from Life Technology. The specific bands around 20 kDa or 70 kDa were sent to Biotech Company (Bei Jing) for mass spectrometry analysis.

### G Quadruplex (G4) Structure Determination

Thioflavin T (ThT) is kind of G4 ligand dye, and its fluorescent signal would increase after binding to G4 structures. So, it is widely used in detecting G4 structure. The Apt-2019 was stained by ThT with the existence of Lit or Kt. Circular dichroism (CD) spectra was used for determination of the subtype of G4 structure.

### Aptamer Synthesis

Aptamer sequences were synthesized by Guang Zhou IGE, HuZhou Hippop and Kunshan Biosyntech company.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Enrichment of Aptamers With High Binding Affinity to LMP1/EBV-Positive Cells After Cell-Selex

With the unique association of EBV infection in NPC pathogenesis, we have discovered aptamers which were enriched from Cell-SELEX using positive and negative pair of NPC43 with (A) LMP1 overexpression or (B) EBV infection.

### (A) Discovery of Aptamers Targeting to LMP1-Expressing Cells:

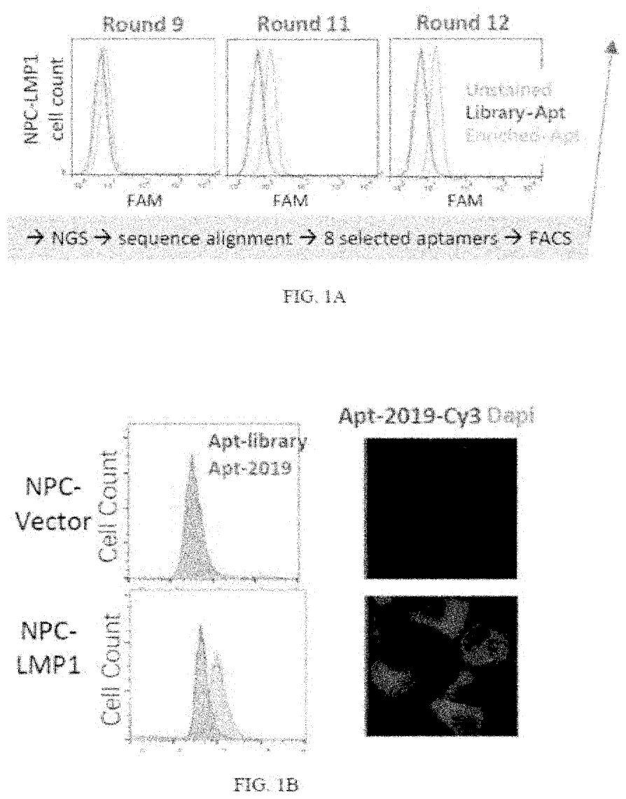

Identification of Apt-2019 which preferentially binds to LMP1-expressing cells. 12 rounds of Cell-SELEX on NPC43^{noEBV} cells with stable expression of LMP1 was performed while vector control cells were used in negative selection. Flow cytometric analyses have shown the gradual increase of fluorescence signals on NPC43^{noEBV}-LMP1 cells bound with the FAM-tagged aptamers enriched from round 9, 11 and 12 Cell-SELEX (FIG. 1A), indicating a successful enrichment process. By next generation sequencing (NGS), the sequences of enriched aptamers were then identified. The top 100 sequences were picked out for multiple sequence alignment by CLUSTALW (see worldwide website: genome.jp/tools-bin/clustalw). 8 sequences were selected out based on the sequence similarity and abundance of sequence reads. Among them, aptamer-2019 (Apt-2019) shows the highest preferential binding to LMP1-expressing cells (FIG. 1B) using flow cytometric analysis and confocal microscopy (FIG. 1B). The specific binding ability of Apt-2019 was also confirmed in multiple LMP1-transfected cell lines including HONE1, C17 and NP460. Apt-2019 could preferentially bind to all these LMP1-overexpressing cells compared to the vector control cells

### Example 2—CCHC-Type Zinc Finger Nucleic Acid Binding Protein (CNBP) is the Binding Target of APT-2019

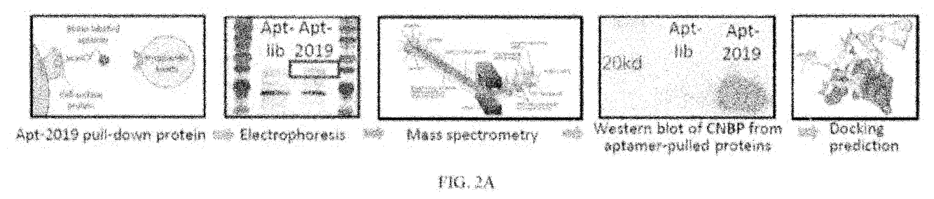

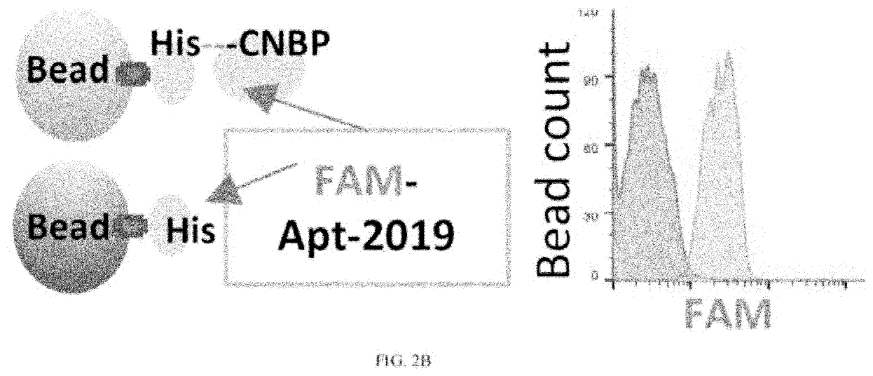

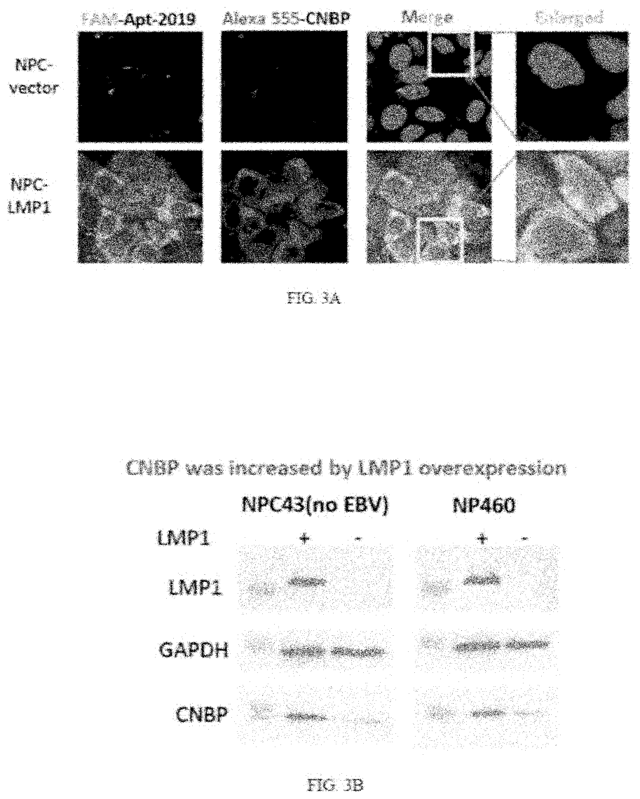

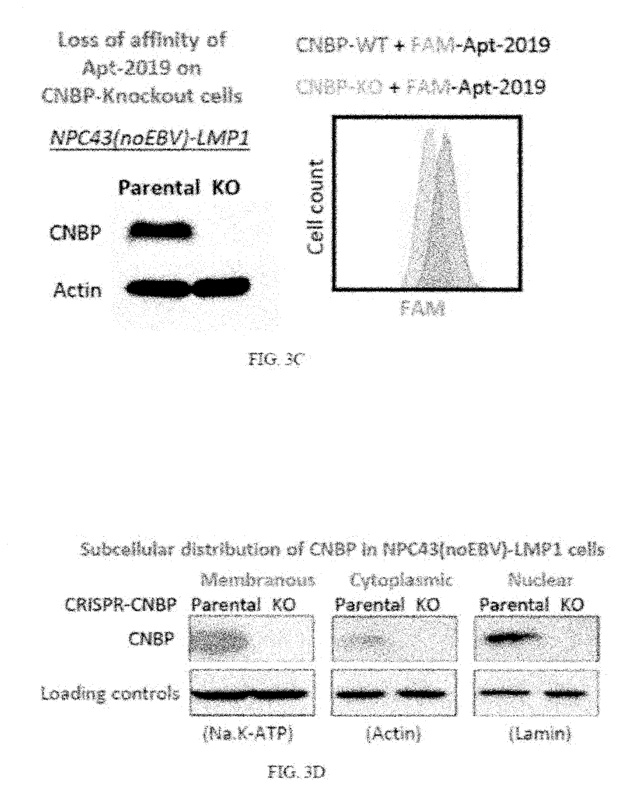

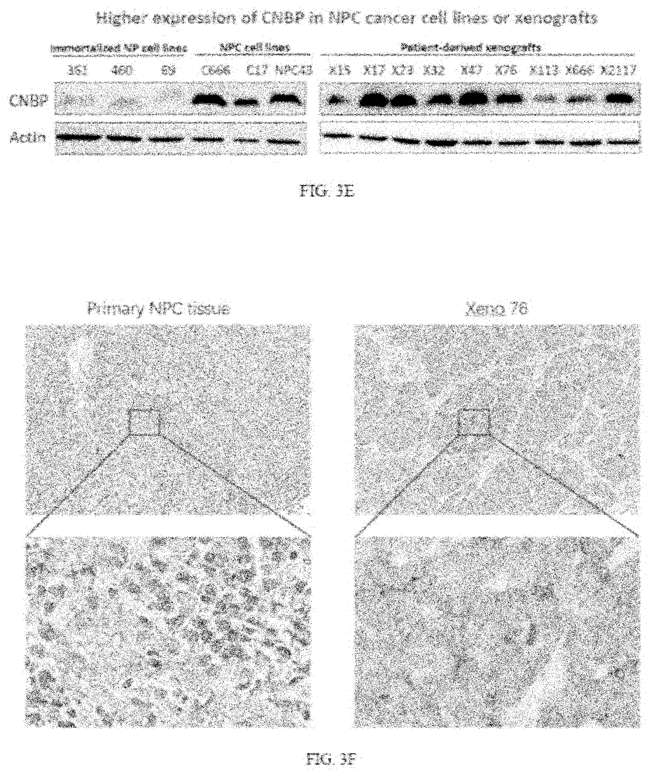

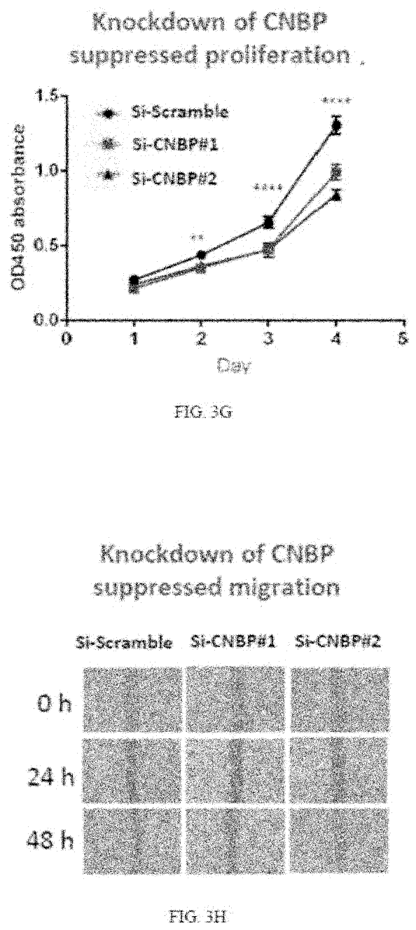

To identify the target surface protein which was bound by the Apt-2019 on LMP1-expressing cells, we have performed pull-down assay using Apt-2019. A specific band around 20 kd was observed in the silver-stained gel after electrophoresis of the pull-down proteins by Apt-2019 when comparing with that of Apt-library (random sequences) (FIG. 2A). The proteins from this band were purified from the gel and subjected for protein identification by tandem mass spectrometry (MS/MS). CNBP was found to be the potential target of Apt-2019. To validate CNBP as the target of Apt-2019, the pull-down proteins from Apt-2019 and Apt-library in LMP1-cell lysates were blotted by the antibody against CNBP (FIG. 2A). The docking model between the Apt-2019 and CNBP was also predicted by HDOCK (sec worldwide website: hdock.phys.hust.edu.cn/) (FIG. 2A). Another validation assay was done by flow analysis of CNBP-tagged and non-tagged beads after binding with Apt-2019-FAM (FIG. 2B). The Apt-2019 could specifically bind to the CNBP-tagged beads. Moreover, Apt-2019-bounded CNBP was specifically expressed on the cell surface, as both the fluorescent signals of CNBP and Apt-2019 were localized on the ‘non-permeabilized’ NPC43-LMP1 cells. (FIG. 3A). Notably, the surface and total expressions of CNBP were upregulated in the cells overexpressing LMP1 (FIGS. 3A-3B). Besides, CRISPR-knockout of CNBP on NPC cells inhibited the binding of Apt-2019 (FIG. 3C). Western blot analysis showed that CNBP was highly expressed in the membrane fraction of NPC43-LMP1 cells (FIG. 3D). All these confirmed Apt-2019 could bind to the cell surface CNBP on NPC cells. Importantly, CNBP was overexpressed in EBV-associated NPC cell lines, PDXs and primary NPC tissues but not in immortalized NP cells (FIGS. 3E-3F). To understand the function of CNBP, we have knocked it down in NPC cells. A decrease in proliferation and migration capability was observed with CNBP knockdown (FIGS. 3G-3H).

### Example 3—Identification of APT-1194 & APT-1798 Which Preferentially Binds to EBV-Positive Cells

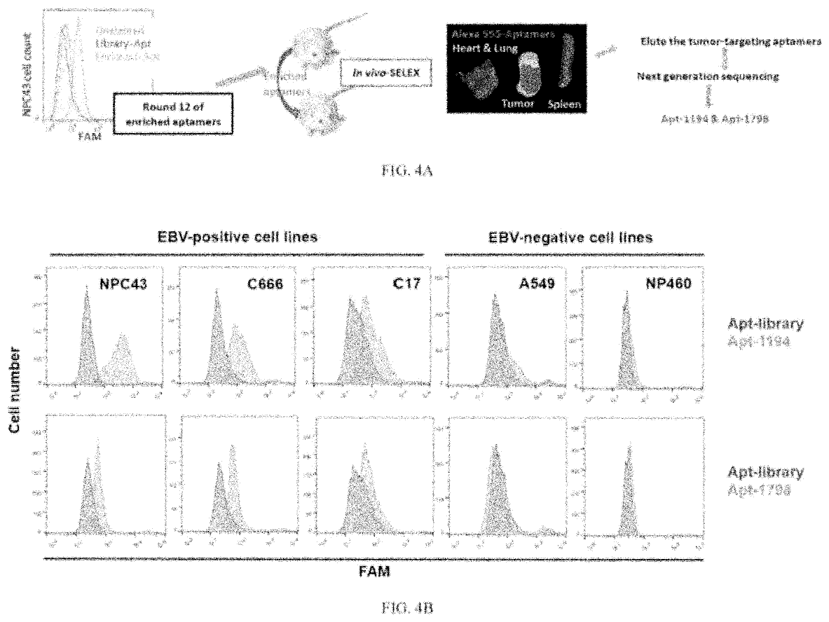

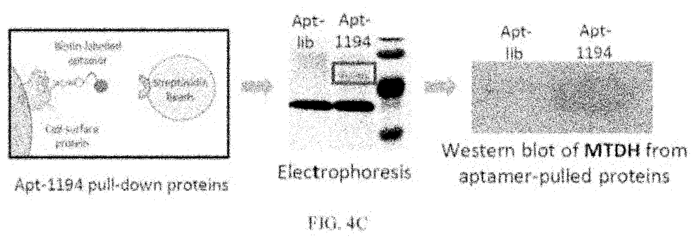

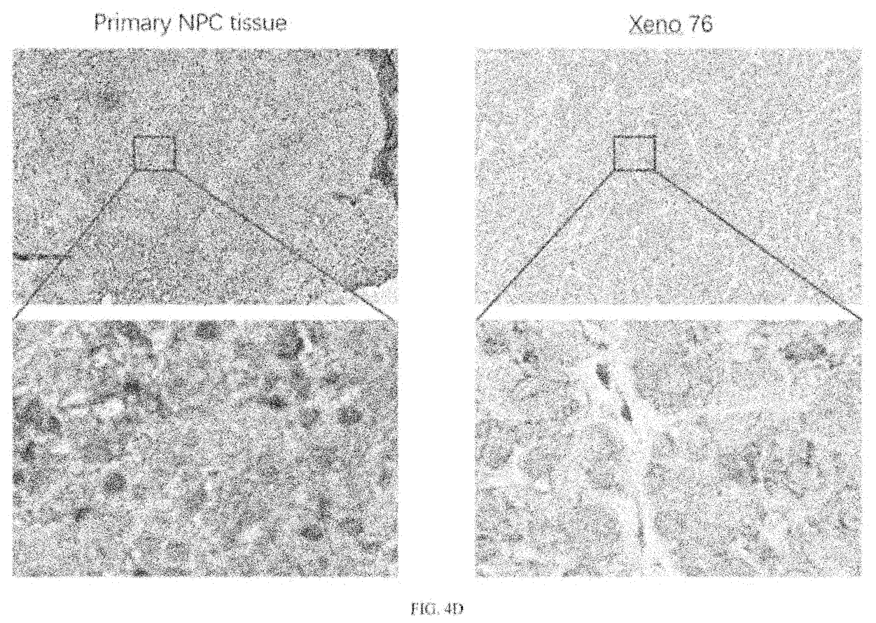

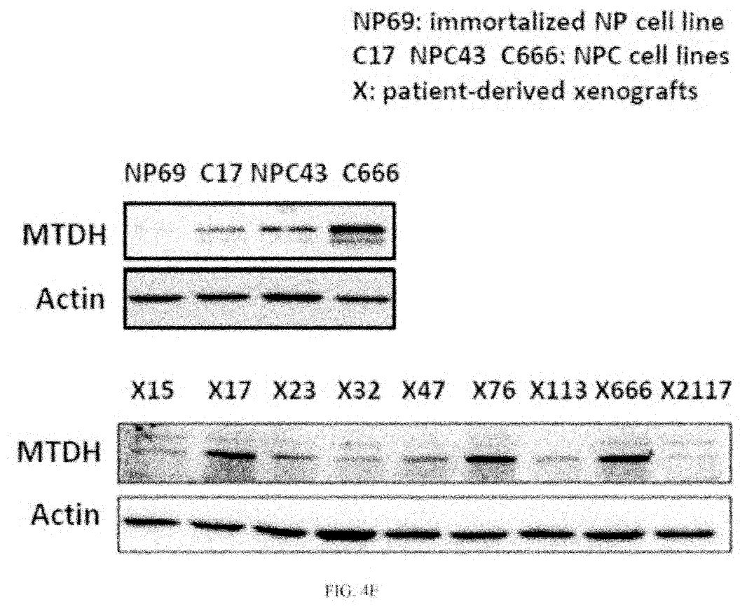

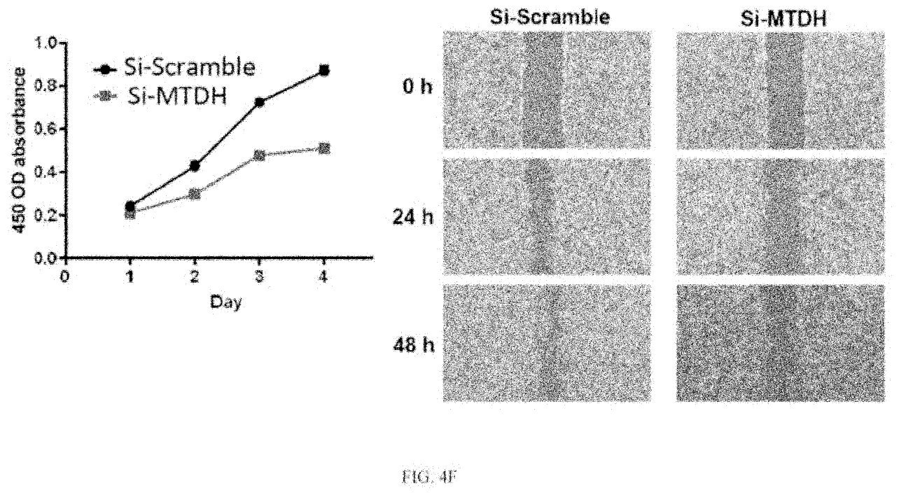

In this part, we have used a combination of Cell-SELEX and in vivo-SELEX to select for cell-surface binding aptamers which can be enriched in EBV-positive NPC tumors in vivo. 12 rounds of Cell-SELEX were performed on a pair of EBV-positive and EBV-negative NPC43 cells ‘NPC43 vs NPC43^{noEBV}’ (FIG. 4A). The enriched aptamers after 12 rounds of Cell-SELEX were then tail vein injected to mice bearing NPC43 subcutaneous tumor. The aptamers accumulated in the tumor were then eluted, amplified by PCR, and re-injected into NPC43-bearing mice (FIG. 4A). The in vivo enriched aptamers were then sent for next generation sequencing. The top two enriched aptamers (Apt-1194 & Apt-1798) were then identified.

### Example 4—Metadherin (MTDH) Is the Binding Target of APT-1194

By binding of Apt-1194 & Apt-1798 with a panel of EBV-positive NPC cell lines (NPC43, C666-1 and C17), EBV-negative lung cancer cell line (A549) and nasopharyngcal cell line (NP460), the specificity of Apt-1194 to EBV-positive cells was confirmed (FIG. 4B). Proteins from NPC43 lysate were pulled down by Apt-1194. After gel electrophoresis, protein purification, and mass spectrometry analysis, MTDH was found to be the target of Apt-1194 as validated by Western blot (FIG. 4C). It has been reported that MTDH is a prognosis marker for NPC (19). We could also observe overexpression of MTDH in NPC cell lines, PDXs and primary NPC tissues (FIGS. 4D-4E), suggesting Apt-1194 can serve as a NPC-targeting aptamer. Knockdown of MTDH could suppress proliferation and migration capability in NPC cells (FIG. 4F)

### Example 5—APT-2019 and APT-1194 Are G-Quadruplex With High Serum Stability and Can be Internalized into Cells Promptly

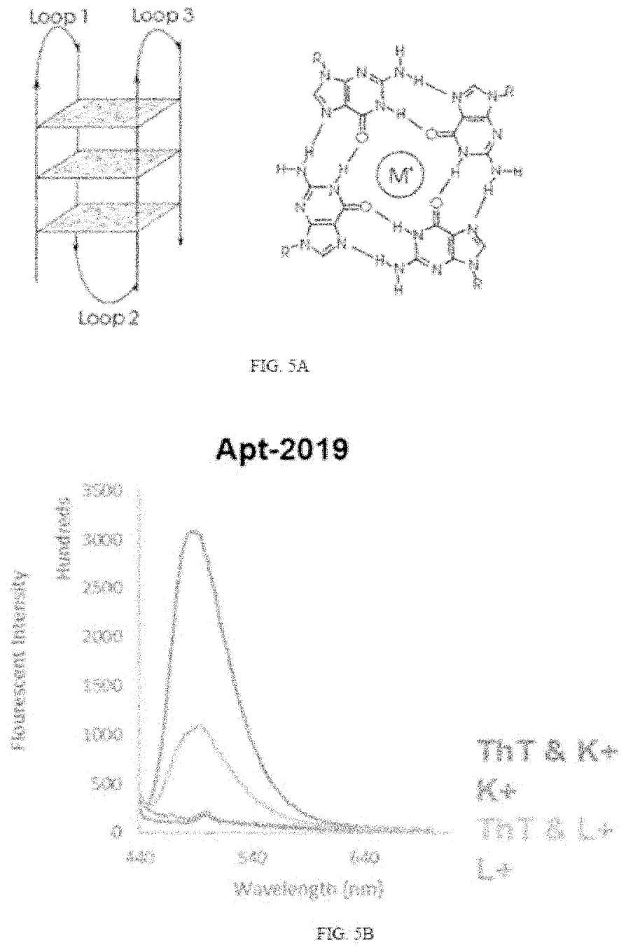

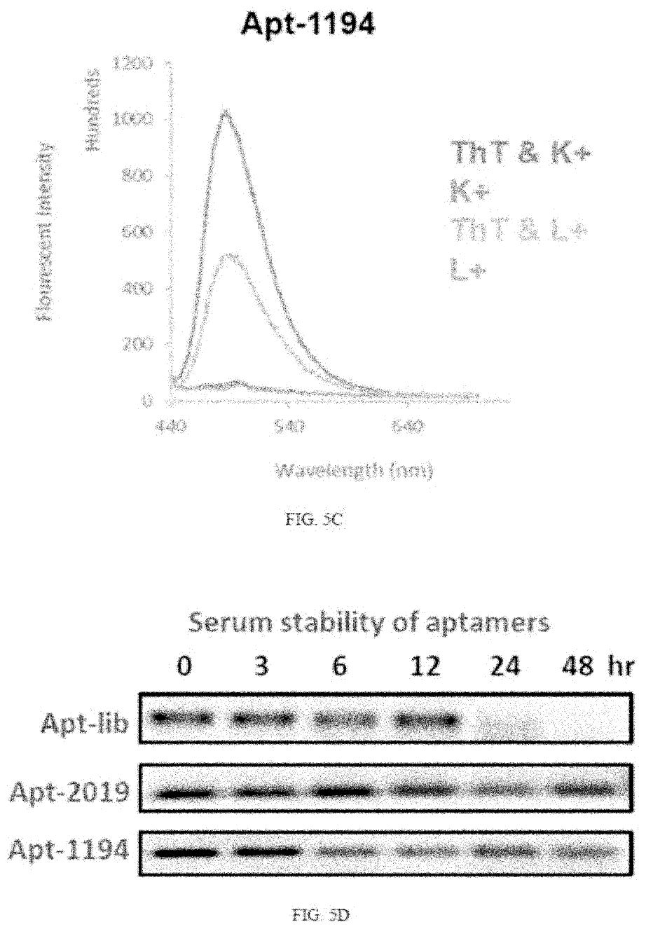

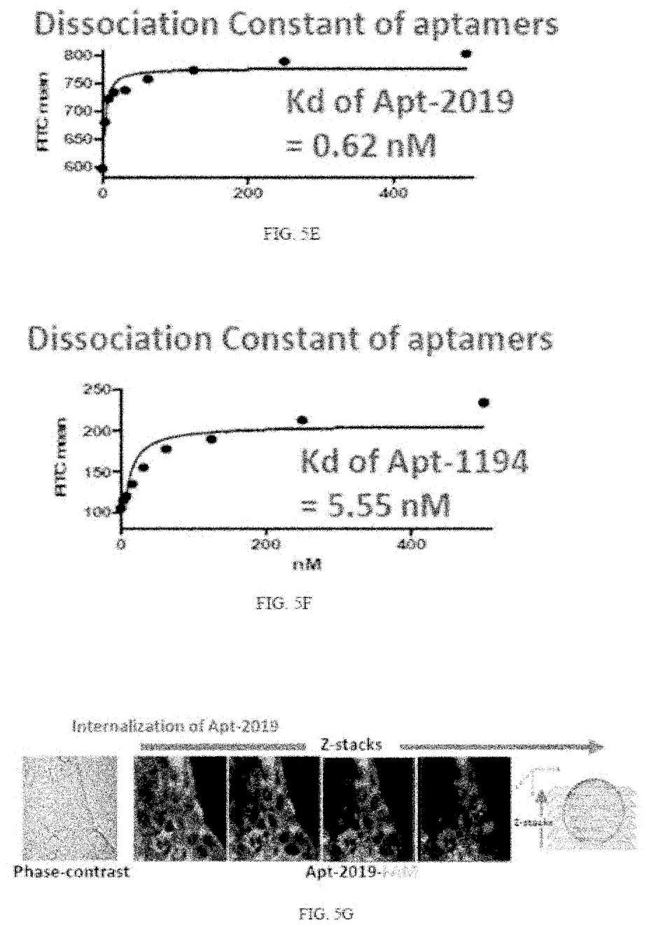

Both Apt-2019 and Apt-1194 are guanine-rich oligonucleotides (GROs) in which the G content are 43 and 41% respectively. GROs can form stable G-quadruplex structures because four guanine bases form a square planar structure called a G-quartet through Hoogsteen base pairing, and these G-quartets spontaneously assemble into four-stranded helical structures termed G-quadruplex (G4) (FIG. 5A) (20). It is well known that G4-forming oligonucleotides are relatively stable against nuclease degradation (21). Therefore, we checked if Apt-2019 and Apt-1194 can form G4 using Thioflavin T (ThT) as a fluorescence light-up probe for G4 formation (22). In the presence of lithium cacodylate buffer or potassium phosphate buffer, both aptamers could fold into G4 which enables the binding and fluorescent emission of ThT ligands (FIGS. 5B-5C). The higher serum stability of Apt-2019 and Apt-1194 than random non-GC rich aptamers (FIG. 5D) can thus be explained by their stable G4 structure. To further characterize these novel NPC-targeting aptamers, we have measured their equilibrium dissociation constant (Kd). Their nanomolar range of Kd indicates a high binding affinity to NPC cells (FIGS. 5E-5F). Importantly, Z-stack confocal microscopy also revealed that Apt-2019 could be internalized into LMP1-expressing NPC43 cells (FIG. 5G). Similar internalization of Apt-1194 could also be observed after incubating with EBV-positive NPC cells.

### Example 6—APT-2019 and APT-1194 as Imaging Probes for NPC Tumors In Vivo

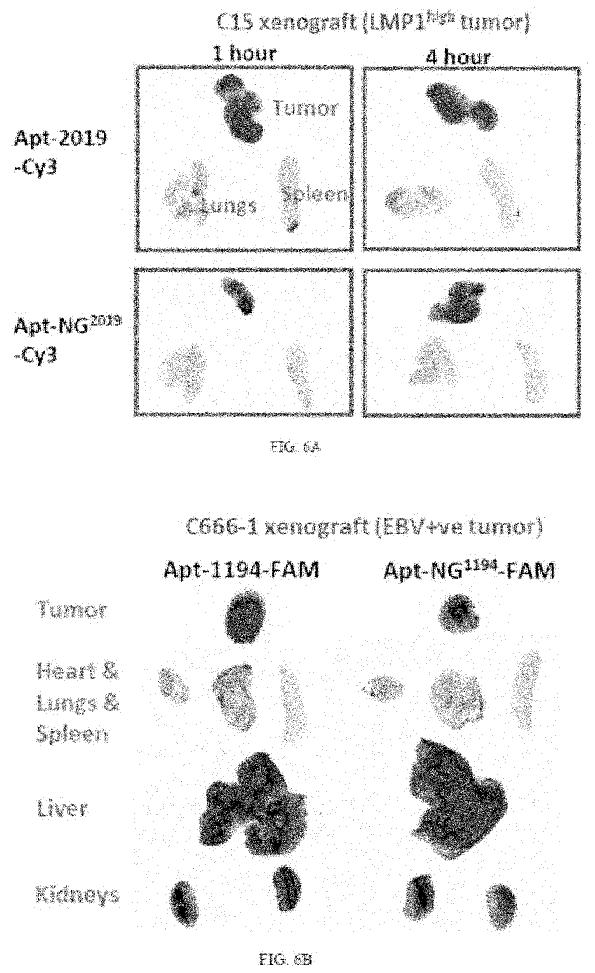

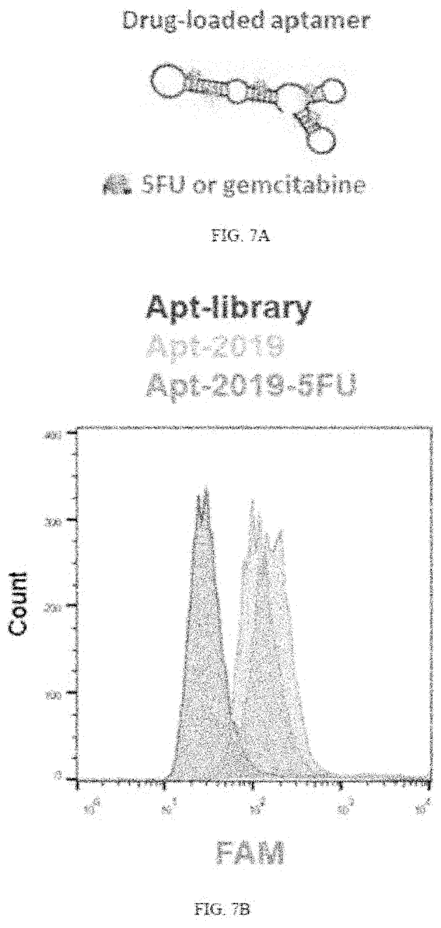

Fluorescence-tagged Apt-2019 and Apt-1194 were tail vein injected in mice bearing C15 tumors (an NPC xenograft with high expression of LMP1 (18)) and EBV-positive C666-1 tumors respectively. We have also generated control aptamers to Apt-2019 and Apt-1194 by replacing all the G to C, so as to destroy the G4-forming ability. The resulting control aptamers are named as Apt-NG²⁰¹⁹ (SEQ ID NO: 4; Apt-NG^{2019 (14-63)} Aptamer: CAGCAGAATCCGTGCCATTCCTCAGACCGGTTCCTGAGTTTGTGATGGAC) and Apt-NG¹¹⁹⁴ (SEQ ID NO:5; Apt-NG^{1194 (1-58)} Aptamer: ATCCAGAGTGACGCAGCACCGAGCGAGTTGGTTTGCTGCCCTGCCCAGAGACCT GGCC). Tumors and organs (e.g., lungs and spleen) were taken out for fluorescence imaging (IVIS Spectrum imager). Representative images demonstrating the accumulation of Apt-2019 or Apt-1194 (but not the control Apt-NG aptamers) in the tumors were shown in FIGS. 6A-6B.

### Example 7—Toxicity of Drug-Loaded Aptamers Which Can Target EBV-Associated NPC Cells

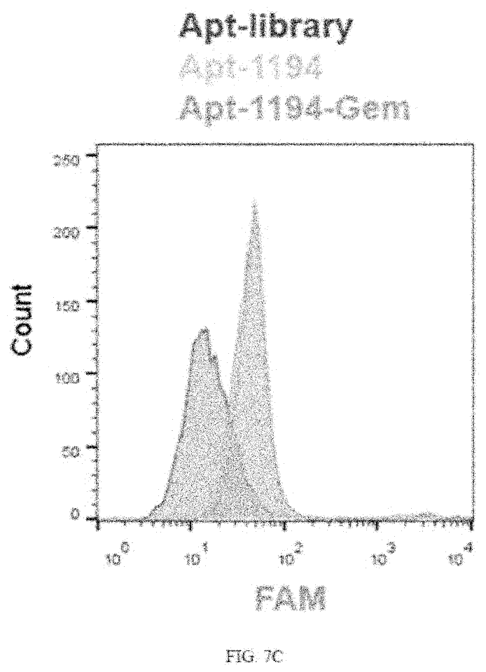

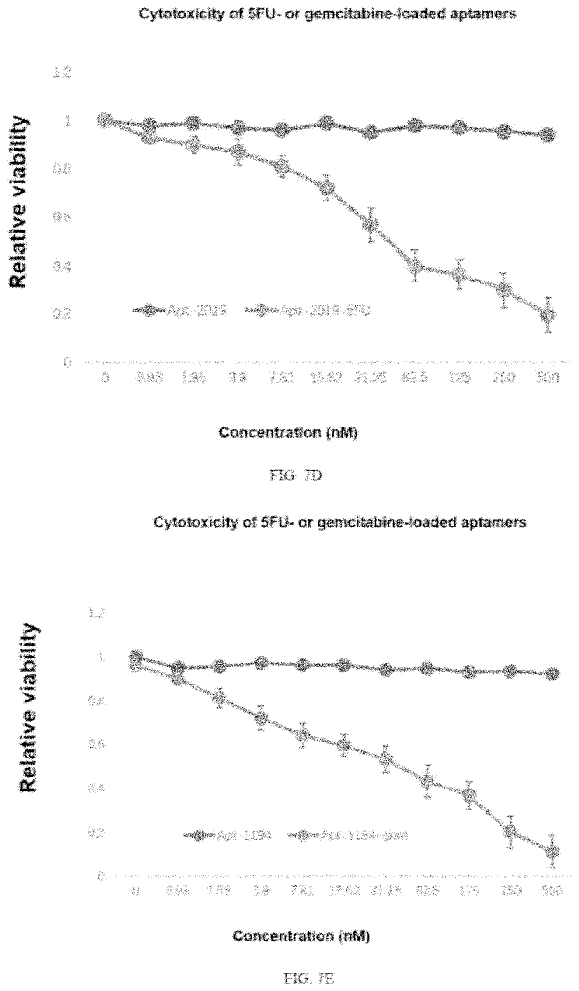

Gemcitabine (Gem) and fluorouracil (5-FU), which have structure similar to that of a natural nucleotide, can be enzymatically incorporated inside DNA strand (FIG. 7A, (23)). These therapeutic drugs can be released in situ after hydrolysis of the aptamers and escape from the lysosome by active nucleotide transporters, e.g., equilibrative nucleoside transporter 3 (ENT3). The conjugation of Gem or 5-FU into our NPC-targeting aptamers create the bifunctional aptamers with both enhanced binding affinity and toxicity. In our preliminary data, we have replaced all the T nucleotides to 5-FU in the Apt-2019, and all the C nucleotides to Gem in the Apt-1194. By doing flow cytometric analysis, the drug-loaded Apt-2019 and Apt-1194 could still preferentially bind to the target cells (FIGS. 7B-7C). Moreover, these drug-loaded aptamers were cytotoxic to the NPC cells with a IC₅₀ around 50 nM (FIGS. 7D-7E).

### Example 8—In Vivo Effective Dose of 5-FU Can be Downregulated by >10 Folds Via Loading into APT-2019

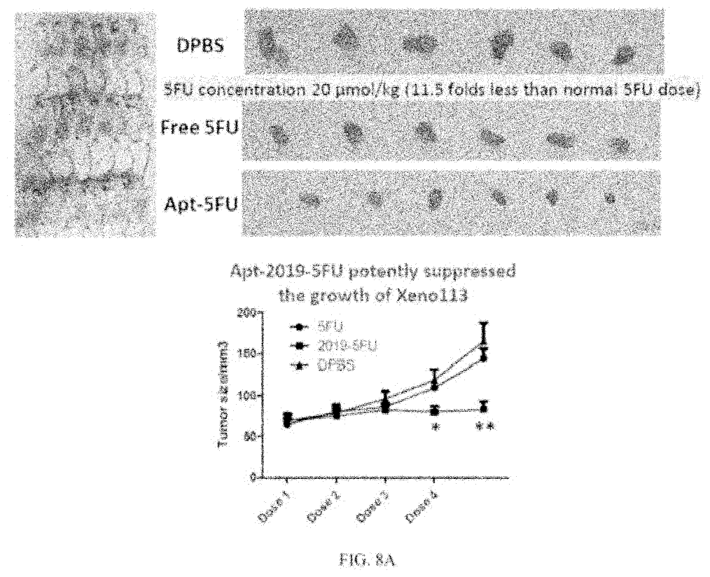

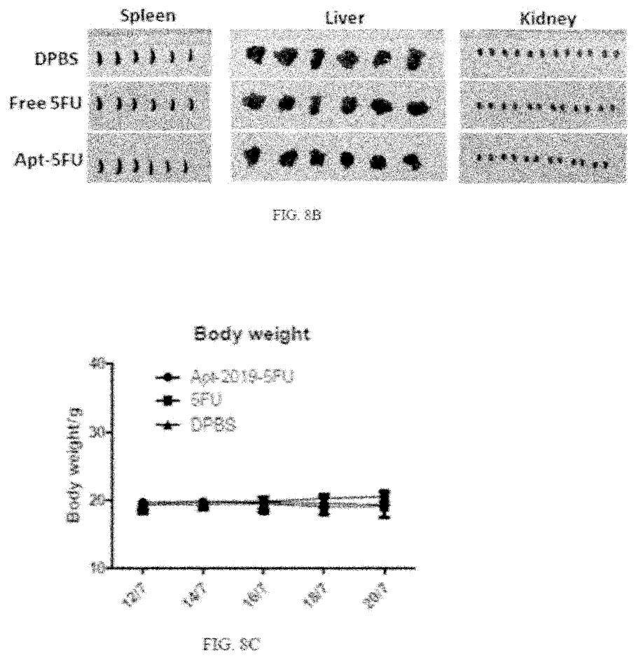

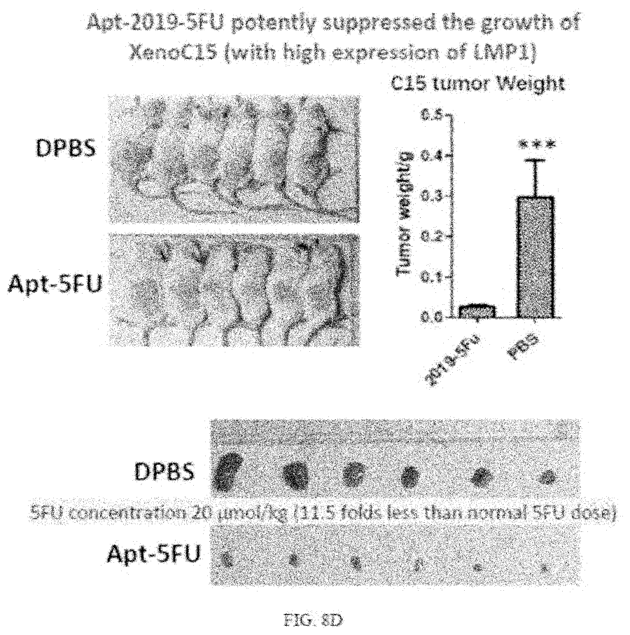

We have examined the in vivo anti-tumor effect of free 5-FU and Apt-2019-5FU when the same dose of 5-FU (20 μmol per kg of mice every two day; 20 μmol 5-FU=2.6 mg 5-FU) was used in these two treatment groups (FIG. 8A). It has to be emphasized that this dose is ˜11.5 folds less than normal effective dose of 5-FU (30 mg/kg/every two day) in treating tumors in mice models (24, 25). Apt-2019-5FU could potently suppress the tumor growth, while free 5-FU could only have minimal effect (FIG. 8A). Importantly, there was no significant change of morphology in liver, spleen kidney and body weight between three groups (FIGS. 8B-8C). Genomic sequence of CNBP was identical in both mice and human. All these suggest that the CNBP-mediated targeting effect of Apt-2019-5FU to NPC tumors did not cause any observable side effects to the treated body. Another similar experiment was done using xenograft-C15, which has high expression of LMP1. The Apt-2019-5FU was also highly effective in inhibiting the tumor growth (FIG. 8D). All these results showed that Apt-2019 could enhance the efficacy of 5-FU by efficiently targeting the 5-FU to the in vivo NPC tumors.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EMBODIMENTS

Embodiment 1. An isolated DNA oligonucleotide aptamer according to SEQ ID NO: 1, 2, or 3, or an oligonucleotide having at least 90% identity to SEQ ID NO: 1, 2, or 3.

Embodiment 2. The DNA oligonucleotide aptamer of embodiment 1, wherein the DNA oligonucleotide aptamer binds to CCHC-Type Zinc Finger Nucleic Acid Binding Protein (CNBP) and/or metadherin (MTDH).

Embodiment 3. The DNA oligonucleotide aptamer of embodiment 1, wherein the DNA oligonucleotide aptamer is conjugated to a fluorescent group, a thiol group, a biotin, or an enzyme.

Embodiment 4. The DNA oligonucleotide aptamer of embodiment 3, wherein the fluorescent group, thiol group, biotin, or enzyme is conjugated to the 5′-terminus of the DNA oligonucleotide aptamer.

Embodiment 5. The DNA oligonucleotide aptamer of embodiment 1, wherein the one or more DNA oligonucleotide aptamers is conjugated to one or more nucleoside analogues or anti-cancer nanoparticles.

Embodiment 6. The DNA oligonucleotide aptamer of embodiment 5, wherein the one or more nucleoside analogues is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine (dideoxyinosine: ddI), zalcitabine (dideoxycytine: ddC), stavudine (d4T), or zidovudine (AZT).

Embodiment 7. The DNA oligonucleotide aptamer of embodiment 5, wherein the anti-cancer nanoparticle comprises an antibody, anti-tumor compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

Embodiment 8. A composition comprising one or more DNA oligonucleotide aptamers according to embodiment 1.

Embodiment 9. The composition of embodiment 8, wherein the one or more DNA oligonucleotide aptamers is conjugated to a fluorescent group, a thiol group, a biotin, or an enzyme.

Embodiment 10. The composition of embodiment 8, wherein the one or more DNA oligonucleotide aptamer is conjugated to one or more nucleoside analogues or anti-cancer nanoparticles.

Embodiment 11. The composition of embodiment 10, wherein the one or more nucleoside analogues is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine, zalcitabine, d4T, or AZT.

Embodiment 12. The composition of embodiment 10, wherein the anti-cancer nanoparticle comprises an antibody, anti-cancer compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

Embodiment 13. The composition of embodiment 8, wherein the composition further comprises a pharmaceutically acceptable carrier and/or excipient.

Embodiment 14. A method of imaging cancer cells in a subject, the method comprising:
i) administering one or more DNA oligonucleotide aptamers according to claim 1 to the subject, wherein the one or more DNA oligonucleotide aptamers is conjugated or linked to a biocompatible imaging probe; and
ii) measuring a level of probe signals from the DNA oligonucleotide aptamer-bound targets.

Embodiment 15. The method of embodiment 14, wherein biocompatible imaging probe is 18F-FDG, 64Cu-NOTA, 11C-methionine, or 11C-choline.

Embodiment 16. The method of embodiment 14, wherein the DNA oligonucleotide aptamers are administered intravenously.

Embodiment 17. The method of embodiment 14, wherein the DNA oligonucleotide aptamers bind to CNBP and/or MTDH.

Embodiment 18. A method of treating cancer, the method comprising administering one or more DNA oligonucleotide aptamers according to embodiment 1 to the subject, wherein the one or more DNA oligonucleotide aptamers is conjugated to a pharmaceutical.

Embodiment 19. The method of embodiment 18, wherein the pharmaceutical is a nucleoside analogue or an anti-cancer nanoparticle.

Embodiment 20. The method of embodiment 19, wherein the nucleoside analogue is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine, zalcitabine, d4T, or AZT.

Embodiment 21. The method of embodiment 19, wherein the anti-cancer nanoparticle comprises an antibody, anti-cancer compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

Embodiment 22. The method of embodiment 18, wherein the cancer is nasopharyngeal carcinoma, breast cancer, pancreatic cancer, bladder cancer, colon and rectal cancer, endometrial cancer, kidney cancer, leukemia, liver cancer, lung cancer, melanoma, or gastric carcinoma.

Embodiment 23. The method of embodiment 18, wherein the DNA oligonucleotide aptamers bind to CNBP and/or MTDH.

### REFERENCES

1. Byun J. Recent Progress and Opportunities for Nucleic Acid Aptamers. Life (Basel). 2021; 11(3).
2. Zhou J, Rossi J. Aptamers as targeted therapeutics: current potential and challenges. Nat Rev Drug Discov. 2017; 16 (3):181-202.
3. Darmostuk M, Rimpelova S, Gbelcova H, Ruml T. Current approaches in SELEX: An update to aptamer selection technology. Biotechnology advances. 2015.
4. Sefah K, Shangguan D, Xiong X, O'Donoghue MB, Tan W. Development of DNA aptamers using Cell-SELEX. Nature protocols. 2010; 5(6):1169-85.
5. Liang S, Kinghorn AB, Voliotis M, Prague JK, Veldhuis JD, Tsaneva-Atanasova K, McArdle CA, Li RHW, Cass AEG, Dhillo WS, Tanner JA. Measuring luteinising hormone pulsatility with a robotic aptamer-enabled electrochemical reader. Nat Commun. 2019; 10(1):852.
6. Cheung YW, Rothlisberger P, Mechaly AE, Weber P, Levi-Acobas F, Lo Y, Wong AWC, Kinghorn AB, Haouz A, Savage GP, Hollenstein M, Tanner JA. Evolution of abiotic cubane chemistries in a nucleic acid aptamer allows selective recognition of a malaria biomarker. Proc Natl Acad Sci U S A. 2020; 117(29):16790-8.
7. Tang MSL, Shiu SC, Godonoga M, Cheung YW, Liang S, Dirkzwager RM, Kinghorn AB, Fraser LA, Heddle JG, Tanner JA. An aptamer-enabled DNA nanobox for protein sensing. Nanomedicine. 2018; 14(4):1161-8.
8. Chen YP, Chan ATC, Le QT, Blanchard P, Sun Y, Ma J. Nasopharyngeal carcinoma. Lancet. 2019; 394(10192):64-80.
9. Rickinson AB, Lo KW. Nasopharyngeal carcinoma: A History. Nasopharyngeal Carcinoma: From Etiology to Clinical Practice Academic Press, London, United Kingdom 2019:1-13.
10. Lee VHF, Lam KO, Lee AW. Standard of care for Nasopharyngeal Carcinoma (2018-2020). Nasopharyngeal Carcinoma: From Etiology to Clinical Practice Academic Press, London, United Kingdom 2019:206-37.
11. Tsang CM, Lui VWY, Bruce JP, Pugh TJ, Lo KW. Translational genomics of nasopharyngeal cancer. Semin Cancer Biol. 2019.
12. Li YY, Chung GT, Lui VW, To KF, Ma BB, Chow C, Woo JK, Yip KY, Seo J, Hui EP, Mak MK, Rusan M, Chau NG, Or YY, Law MH, Law PP, Liu ZW, Ngan HL, Hau PM, Verhoeft KR, Poon PH, Yoo SK, Shin JY, Lee SD, Lun SW, Jia L, Chan AW, Chan JY, Lai PB, Fung CY, Hung ST, Wang L, Chang AM, Chiosea SI, Hedberg ML, Tsao SW, van Hasselt AC, Chan AT, Grandis JR, Hammerman PS, Lo KW. Exome and genome sequencing of nasopharynx cancer identifies NF-kappaB pathway activating mutations. Nat Commun. 2017; 8:14121.
13. Lin DC, Meng X, Hazawa M, Nagata Y, Varela AM, Xu L, Sato Y, Liu LZ, Ding LW, Sharma A, Goh BC, Lee SC, Petersson BF, Yu FG, Macary P, Oo MZ, Ha CS, Yang H, Ogawa S, Loh KS, Koeffler HP. The genomic landscape of nasopharyngeal carcinoma. Nat Genet. 2014; 46(8):866-71.
14. Dai W, Zheng H, Cheung AK, Tang CS, Ko JM, Wong BW, Leong MM, Sham PC, Cheung F, Kwong DL, Ngan RK, Ng WT, Yau CC, Pan J, Peng X, Tung S, Zhang Z, Ji M, Chiang AK, Lee AW, Lee VH, Lam KO, Au KH, Cheng HC, Yiu HH, Lung ML. Whole-exome sequencing identifies MSTIR as a genetic susceptibility gene in nasopharyngeal carcinoma. Proc Natl Acad Sci U S A. 2016; 113(12):3317-22.
15. Bruce JP, To KF, Lui VWY, Chung GTY, Chan YY, Tsang CM, Yip KY, Ma BBY, Woo JKS, Hui EP, Mak MKF, Lee SD, Chow C, Velapasamy S, Or YYY, Siu PK, El Ghamrasni S, Prokopec S, Wu M, Kwan JSH, Liu Y, Chan JYK, van Hasselt CA, Young LS, Dawson CW, Paterson IC, Yap LF, Tsao SW, Liu FF, Chan ATC, Pugh TJ, Lo KW. Whole-genome profiling of nasopharyngeal carcinoma reveals viral-host co-operation in inflammatory NF-kappaB activation and immune escape. Nat Commun. 2021; 12(1):4193.
16. Tsao SW, Tsang CM, Lo KW. Epstein-Barr virus infection and nasopharyngeal carcinoma. Philos Trans R Soc Lond B Biol Sci. 2017; 372(1732).
17. Dawson CW, Port RJ, Young LS. The role of the EBV-encoded latent membrane proteins LMP1 and LMP2 in the pathogenesis of nasopharyngeal carcinoma (NPC). Semin Cancer Biol. 2012; 22(2):144-53.
18. Tsao SW, Tsang CM, To KF, Lo KW. The role of Epstein-Barr virus in epithelial malignancies. The Journal of pathology. 2015; 235(2):323-33.
19. Hui AB, Bruce JP, Alajez. NM, Shi W, Yue S, Perez-Ordonez. B, Xu W, O'Sullivan B, Waldron J, Cummings B, Gullane P, Siu L, Liu FF. Significance of dysregulated metadherin and microRNA-375 in head and neck cancer. Clin Cancer Res. 2011; 17(24):7539-50.
20. Rhodes D, Lipps HJ. G-quadruplexes and their regulatory roles in biology. Nucleic Acids Res. 2015; 43(18):8627-37.
21. Bishop JS, Guy-Caffey JK, Ojwang JO, Smith SR, Hogan ME, Cossum PA, Rando RF, Chaudhary N. Intramolecular G-quartet motifs confer nuclease resistance to a potent anti-HIV oligonucleotide. J Biol Chem. 1996; 271(10):5698-703.
22. Renaud de la Faverie A, Guedin A, Bedrat A, Yatsunyk LA, Mergny JL. Thioflavin T as a fluorescence light-up probe for G4 formation. Nucleic Acids Res. 2014; 42(8):e65.
23. Xuan W, Peng Y, Deng Z, Peng T, Kuai H, Li Y, He J, Jin C, Liu Y, Wang R, Tan W. A basic insight into aptamer-drug conjugates (ApDCs). Biomaterials. 2018; 182:216-26.
24. Lu Y, Huang H, Yang H, Chen D, Wu S, Jiang Z, Wang R. Small molecule inhibitor TW-37 is tolerable and synergistic with chemotherapy in nasopharyngeal carcinoma. Cell Cycle. 2017; 16(14):1376-83.
25. Zhang P, Lai ZL, Chen HF, Zhang M, Wang A, Jia T, Sun WQ, Zhu XM, Chen XF, Zhao Z, Zhang J. Curcumin synergizes with 5-fluorouracil by impairing AMPK/ULK1-dependent autophagy, AKT activity and enhancing apoptosis in colon cancer cells with tumor growth inhibition in xenograft mice. J Exp Clin Cancer Res. 2017; 36(1):190.

1. An isolated DNA oligonucleotide aptamer according to SEQ ID NO: 1, 2, or 3, or an oligonucleotide having at least 90% identity to SEQ ID NO: 1, 2, or 3.

2. The DNA oligonucleotide aptamer of claim 1, wherein the DNA oligonucleotide aptamer binds to CCHC-Type Zinc Finger Nucleic Acid Binding Protein (CNBP) and/or metadherin (MTDH).

3. The DNA oligonucleotide aptamer of claim 1, wherein the DNA oligonucleotide aptamer is conjugated to a fluorescent group, a thiol group, a biotin, or an enzyme.

4. The DNA oligonucleotide aptamer of claim 3, wherein the fluorescent group, thiol group, biotin, or enzyme is conjugated to the 5′-terminus of the DNA oligonucleotide aptamer.

5. The DNA oligonucleotide aptamer of claim 1, wherein the DNA oligonucleotide aptamer is conjugated to a nucleoside analogue or an anti-cancer nanoparticle.

6. The DNA oligonucleotide aptamer of claim 5, wherein the nucleoside analogue is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine (dideoxyinosine: ddI), zalcitabine (dideoxycytine: ddC), stavudine (d4T), or zidovudine (AZT).

7. The DNA oligonucleotide aptamer of claim 5, wherein the anti-cancer nanoparticle comprises an antibody, anti-tumor compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

8. A composition comprising a DNA oligonucleotide aptamer according to claim 1.

9. The composition of claim 8, wherein the DNA oligonucleotide aptamer is conjugated to a fluorescent group, a thiol group, a biotin, or an enzyme.

10. The composition of claim 8, wherein the DNA oligonucleotide aptamer is conjugated to a nucleoside analogue or an anti-cancer nanoparticle.

11. The composition of claim 10, wherein the nucleoside analogue is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine, zalcitabine, d4T, or AZT.

12. The composition of claim 10, wherein the anti-cancer nanoparticle comprises an antibody, anti-cancer compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

13. The composition of claim 8, wherein the composition further comprises a pharmaceutically acceptable carrier and/or excipient.

14. A method of imaging cancer cells in a subject, the method comprising:
i) administering a plurality of DNA oligonucleotide aptamers according to claim 1 to the subject, wherein the plurality of DNA oligonucleotide aptamers each is conjugated or linked to a biocompatible imaging probe; and
ii) measuring a level of probe signals from cells to which the DNA oligonucleotide aptamers are bound.

15. The method of claim 14, wherein the biocompatible imaging probe is 18F-FDG, 64Cu-NOTA, 11C-methionine, or 11C-choline.

16. The method of claim 14, wherein the DNA oligonucleotide aptamers are administered intravenously.

17. The method of claim 14, wherein the DNA oligonucleotide aptamers bind to CNBP and/or MTDH.

18. A method of treating cancer in a subject, the method comprising administering a plurality of DNA oligonucleotide aptamers according to claim 1 to the subject, wherein the plurality of DNA oligonucleotide aptamers each is conjugated to a pharmaceutical.

19. The method of claim 18, wherein the pharmaceutical is a nucleoside analogue or an anti-cancer nanoparticle.

20. The method of claim 19, wherein the nucleoside analogue is 5-FU, gemcitabine, cytarabine, mercaptopurine, azacytidine, cladribine, decitabine, fluorouracil, floxuridine, fludarabine, nelarabine, azathioprine, allopurinol, trimethoprim, lamivudine, emtricitabine, telbivudine, tenofovir, adefovir, fialuridine, didanosine, zalcitabine, d4T, or AZT.

21. The method of claim 19, wherein the anti-cancer nanoparticle comprises an antibody, anti-cancer compound, siRNA, alkylating agent, platinum coordination complex, antibiotic, antifolate, purine analogue, pyrimidine analogue, biologic response modifier, histone deacetylase inhibitor, Antiandrogen, Antiestrogen, Gonadotropin Releasing Hormone Analogue, Peptide Hormone, Protein Kinase Inhibitor, Topoisomerase Inhibitor, Vinca Alkaloid, Asparaginase, Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, Venetoclax, or a combination of any of them.

22. The method of claim 18, wherein the cancer is nasopharyngeal carcinoma, breast cancer, pancreatic cancer, bladder cancer, colon and rectal cancer, endometrial cancer, kidney cancer, leukemia, liver cancer, lung cancer, melanoma, or gastric carcinoma.

23. The method of claim 18, wherein the DNA oligonucleotide aptamers bind to CNBP and/or MTDH.
